(19) United States
(12) Plant Patent Application Publication
(10) Pub. No.: US 2016/0270276 P1
(43) Pub. Date: Sep. 15, 2016

(54) Strawberry plant named 'DRISSTRAWFORTYFIVE'

(50) ;

(71) Applicant: DRISCOLL STRAWBERRY ASSOCIATES, INC., WATSONVILLE, CA (US)

(72) Inventors: MATTHIAS D. VITTEN, APTOS, CA (US); CARLOS D. FEAR, WEST MALLING (GB); KATALIN PAKOZDI, MAIDSTONE (GB)

(21) Appl. No.: 14/544,902

(22) Filed: Mar. 6, 2015

(51) Int. Cl. A01H 5/00

(52) U.S. Cl. PLT/208

(57) ABSTRACT

A new and distinct variety of strawberry plant named ‘DrisStrawFortyFive’ particularly characterized by medium red, sweet fruit, early harvest maturity and moderate resistance to powdery mildew, is disclosed.

### GENUS AND SPECIES

Fragaria×ananassa

### VARIETY DENOMINATION

‘DrisStrawFortyFive’

### BACKGROUND OF THE NEW PLANT

The present invention relates to a new and distinct strawberry variety designated ‘DrisStrawFortyFive’ and botanically known as Fragaria×ananassa. This new strawberry variety was discovered in Kent, United Kingdom in June 2010 and originated from a cross between the proprietary female parent ‘DrisStrawThirtyTwo’ (U.S. Plant Pat. No. 24,333) and the proprietary male parent ‘KGEM 0629-001’ (unpatented). A single plant was selected and asexually propagated via tissue culture and vegetative cuttings in Kent, United Kingdom in 2010.

‘DrisStrawFortyFive’ underwent further testing in Kent, United Kingdom from 2010-2014. The present invention has been found to retain its distinctive characteristics through successive asexual propagations via stolons and tissue culture.

Plant Breeder's Rights for this variety have not been applied for. ‘DrisStrawFortyFive’ has not been made publicly available or sold anywhere in the world more than one year prior to the filing date of this application.

### SUMMARY OF THE INVENTION

The following are the most outstanding and distinguishing characteristics of this new cultivar when grown under normal horticultural practices in Kent, United Kingdom.

1. Medium red, sweet fruit;

2. Early harvest maturity; and

3. Moderate resistance to powdery mildew.

### DESCRIPTION OF THE PHOTOGRAPHS

The accompanying color photographs show typical specimens of the new variety at various stages of development. The colors shown are as true as can be reasonably obtained by conventional photographic procedures. The photographs were taken from ten to twelve-month-old plants.

FIG. 1 shows upper and lower surfaces of the leaves of the plant with three leaflets.

FIG. 2 shows both upper and lower surfaces of the flowers.

FIG. 3 shows the whole fruit.

FIG. 4 shows the fruit in longitudinal cross-section.

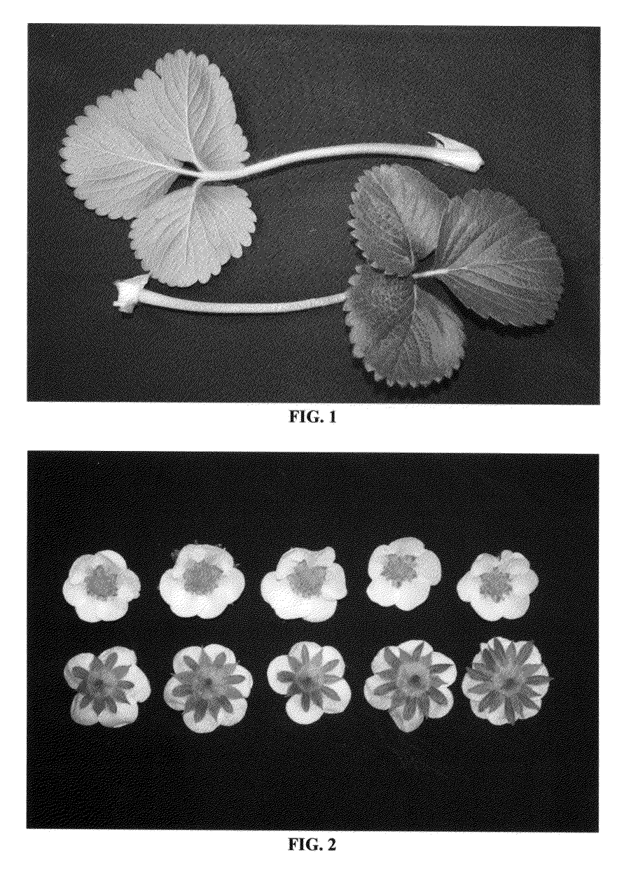

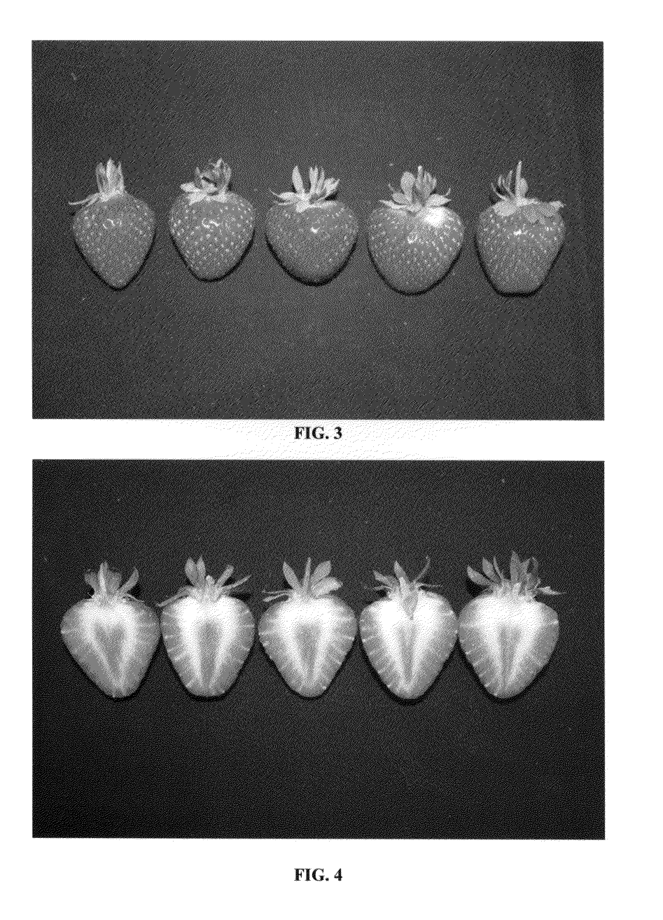

### DESCRIPTION OF THE NEW VARIETY

The following detailed descriptions set forth the distinctive characteristics of ‘DrisStrawFortyFive’. The data which define these characteristics is based on observations taken in Kent, United Kingdom from 2010 to 2014. This description is in accordance with UPOV terminology. Color designations, color descriptions, and other phenotypical descriptions may deviate from the stated values and descriptions depending upon variation in environmental, seasonal, climatic, and cultural conditions. ‘DrisStrawFortyFive’ has not been observed under all possible environmental conditions. The botanical description of ‘DrisStrawFortyFive’ was taken from ten to twelve-month-old plants. Color references are primarily to the R.H.S. Colour Chart of The Royal Horticultural Society of London (R.H.S.) (2007 edition). Descriptive terminology follows the Plant Identification Terminology, An Illustrated Glossary, 2^{nd} edition by James G. Harris and Melinda Woolf Harris, unless where otherwise defined.

### DETAILED BOTANICAL DESCRIPTION OF THE PLANT

Classification:
   Species.—Fragaria×ananassa.
   Common name.—Strawberry.
   Denomination.—‘DrisStrawFortyFive’.
Parentage:
   Female parent.—The proprietary variety ‘DrisStrawThirtyTwo’ (U.S. Plant Pat. No. 24,333).
   Male parent.—The proprietary variety ‘KGEM 0629-001’ (unpatented).
Plant:
   Height.—47.1 cm.
   Diameter.—56.7 cm.
   Number of crowns/plant.—4.
   Habit.—Upright.
   Density of individual plant.—Medium.
   Vigor (health and hardiness of plant).—Medium.
Terminal leaflets:
   Size.—Medium. Length: 10.8 cm Width: 9.9 cm Length/width ratio: 1.1 (Longer than broad).
   Number of teeth/terminal leaflet.—20.
   Shape of teeth.—Obtuse — serrate to crenate.
   Color.—Upper surface: RHS N137C (Dark green) Lower surface: RHS 137C (Medium green).
   Shape in cross section.—Slightly concave.
   Blistering.—Weak.
   Glossiness.—Medium.
   Number of leaflets.—Three only.
   Shape.—Orbicular.
   Base shape.—Obtuse.
   Apex descriptor.—Convex.
   Margin.—Serrate.
   Margin profile.—Revolute (margins rolled backwards).
   Variegation.—Absent.
Petiole:
   Length.—Medium; 32.9 cm.
   Diameter.—8.31 mm.
   Pubescence.—Medium.
   Pose of hairs.—Slightly upwards.
   Color.—RHS 144B (Medium yellow-green).
   Bract frequency.—0.
Petiolule:
   Length.—8.31 mm.
   Diameter.—2.94 mm.
   Color.—RHS N144C (Medium yellow-green).
Stipule:
   Length.—39.4 mm.
   Width.—11.13 mm.
   Pubescence.—Medium.
   Stipule anthocyanin coloration.—Weak; RHS 63C (Medium red-purple).
Stolons:
   Number of stolons.—Many.
   Average number of daughter plants (per square meter).—114.
   Anthocyanin coloration.—Absent or very weak; RHS 144C (Medium yellow-green).
   Diameter at bract.—3.31 mm.
   Thickness.—Thick.
   Density of pubescence.—Sparse.
Inflorescence:
   Position relative to foliage.—Level.
   Number of flowers per plant.—Many.
   Time of flowering (50% of plants at first flower).—Early.
   Flower size.—Medium.
   Flower diameter.—29.44 mm.
   Petals.—Shape: Orbicular Apex: Rounded Base: Concavo-convex Margin: Entire Spacing: Overlapping Length: 12.93 mm Width: 14.20 mm Length/width ratio: 0.9 (Broader than long) Petal number per flower: 5 Color (upper surface): RHS NN155C (White).
   Calyx.—Diameter: 26.65 mm Diameter relative to corolla: Smaller Inner calyx diameter relative to outer: Same size Insertion of calyx: Level Pose of calyx segments: Spreading — outwards Size of calyx in relation to fruit: Slightly smaller Adherence of calyx: Medium.
   Sepal.—Shape: Ovate Apex: Acuminate Margin: Entire Length: 9.11 mm Width: 4.99 mm Sepal number: 5.
   Receptacle color.—RHS 12B (Medium yellow).
   Stamen.—Present Anther color: RHS 153C (Medium yellow-green).
   Pedicel.—Attitude of hairs: Slightly upwards.
Fruiting truss:
   Length.—Long; 42.4 cm.
   Diameter at base of truss.—5.98 mm.
   Number of berries per fruiting truss.—11.
   Attitude at first picking.—Semi-erect.
   Color at base of truss.—RHS 144B (Medium yellow-green).
Fruit:
   Relative fruit size.—Medium.
   Length.—41.54 mm.
   Width.—40.15 mm.
   Length/width ratio.—1.0 (As long as broad).
   Fruit hollow length.—30.47 mm.
   Fruit hollow width.—15.42 mm.
   Fruit hollow length/width ratio.—2.0 (Longer than broad).
   Fruit hollow center (cavity).—Medium.
   Fruit weight.—23.0 g.
   Predominant fruit shape.—Conical.
   Difference in shape between primary and secondary fruits.—None or very slight.
   Evenness of fruit surface.—Even or very slightly uneven.
   Fruit skin color.—RHS 42B (Medium red).
   Evenness of fruit color.—Even or very slightly uneven.
   Fruit glossiness.—Medium.
   Achenes.—Insertion of achenes: Below surface Coloration (sunward side of berry): RHS N167A (Medium greyed-orange) Coloration (shaded side of berry): RHS N167C (Medium greyed-orange) Number per berry: 262 Weight (weight of achenes divided by total # seed): 0.0086 g Width of band without achenes: Absent or very narrow.
   Firmness of flesh (when fully ripe).—Medium.
   Color of flesh (excluding core).—RHS 40B (Medium red).
   Color of core.—RHS 40C (Medium red).
   Evenness of flesh color.—Even.
   Distribution of flesh color.—Marginal and central.
   Sweetness.—Strong.
   Acidity.—Weak.
   Texture when tasted.—Fine.
   Type of bearing.—Not everbearing — not remontant.
   Harvest interval.—Late May-mid-July.
   Harvest maturity.—Early.
   Production.—870 grams per plant in Class I & II UK (2012-2014).
Disease and pest resistance:
   Powdery mildew.—Moderately resistant.
   Verticillium wilt.—Moderately susceptible.

### COMPARISON WITH PARENTAL AND COMMERCIAL VARIETIES

When ‘DrisStrawFortyFive’ is compared to the female parent ‘DrisStrawThirtyTwo’ (U.S. Plant Pat. No. 24,333), ‘DrisStrawFortyFive’ is higher yielding, lighter red color, and better flavor than ‘DrisStrawThirtyTwo’.

When ‘DrisStrawFortyFive’ is compared to the male parent ‘KGEM 0629-001’ (unpatented), ‘DrisStrawFortyFive’ has fruit that is lighter red color and slightly larger in size than ‘KGEM 0629-001’.

‘DrisStrawFortyFive’ differs from the commercial variety ‘Sonata’ (U.S. Plant Pat. No. 18,000), in that ‘DrisStrawFortyFive’ has orbicular shaped petals having a concavo-convex base, whereas ‘Sonata’ has broadly ovate shaped petals having an attenuate base. Additionally, ‘DrisStrawFortyFive’ has a medium-sized fruit hollow center and achenes that are inserted below the fruit surface, whereas ‘Sonata’ has an absent or weakly expressed fruit hollow center and achenes that are inserted level with the fruit surface.

‘DrisStrawFortyFive’ differs from the commercial variety ‘Elsanta’ (unpatented), in that ‘DrisStrawFortyFive’ has medium-sized fruit with medium flesh firmness and a medium red colored core, whereas ‘Elsanta’ has large, firm fruit with a white colored core.

We claim:

1. A new and distinct variety of strawberry plant named ‘DrisStrawFortyFive’, substantially as illustrated and described herein.
